(19) United States
(12) Patent Application Publication
(10) Pub. No.: US 2004/0241701 A1
(43) Pub. Date: Dec. 2, 2004

(54) Method for generation of modular polynucleotides using solid supports

(76) Inventor: Vaughn Smider, Alameda, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/494,996

(22) PCT Filed: Nov. 7, 2002
(86) PCT No.: PCT/US02/36170

Related U.S. Application Data
(60) Provisional application No. 60/337,718, filed on Nov. 7, 2001.

(51) Int. Cl. C12Q 1/68; G01N 33/53

(52) U.S. Cl. 435/6; 435/7.1

(57) ABSTRACT

The invention generally relates to nucleic acid cloning and genetic engineering. The invention also relates to the field of molecular evolution and protein engineering.

### CROSS-REFERENCES TO RELATED APPLICATIONS

This application claims the benefit of U.S. Provisional Application No. 60/337,718 filed Nov. 7, 2001, which is herein incorporated by reference.

### TECHNICAL FIELD OF THE INVENTION

The invention generally relates to nucleic acid cloning and genetic engineering. The invention also relates to the field of molecular evolution and protein engineering.

### BACKGROUND OF THE INVENTION

Recombinant DNA refers to the covalent attachment of DNA molecules to one another that would normally not be coupled in nature. Generally, recombinant DNA is produced through the linking of at least two genetic elements to one another. The linkage of two DNA molecules is often accomplished by incubating the genetic elements together with a DNA ligase under the appropriate conditions. Alternatively, genetic elements can be linked in vivo using the recombinational and repair apparatus that is present within cells. Recombinant molecules can also be produced by linking two polynucleotides together via an oligonucleotide “bridge”, such that extension with a polymerase and ligation of a nick produce a new molecule comprising the original genetic elements. Also, genetic elements can be linked through the use of a variant of the polymerase chain reaction (PCR) called DNA shuffling, whereby two or more genetic elements that are homologous are fragmented, denatured, annealed, and extended with polymerase to produce hybrid molecules.

The classic method of de nova gene synthesis entails sequential annealing (hybridization) and ligation of the component synthetic oligonucleotides, a few at a time, in a homogeneous aqueous solution (Khorana, Science (1979) 203: 614-25). In this method, a mixture of overlapping, complementary oligonucleotides are annealed under conditions that favor formation of a correct double-stranded fragment (duplex DNA) with strand interruptions (nicks) at adjacent positions along the two strands. The resultant construct is then isolated and submitted to subsequent rounds of annealing, ligation, and isolation. The method requires efficient, rapid, and specific hybridization, the chemical synthesis of all the components of the gene, and many analytical and purification operations.

Short polynucleotides can also be produced by oligonucleotide synthesis on a solid support. Oligonucleotide synthesis proceeds via linear coupling of individual monomers in a stepwise reaction. The reactions are generally performed on a solid phase support by first coupling the 3′ end of the first monomer to the support. The second monomer is added to the 5′ end of the first monomer in a condensation reaction to yield a dinucleotide coupled to the solid support. At the end of each coupling reaction, the by-products and unreacted, free monomers are washed away so that the starting material for the next round of synthesis is the pure oligonucleotide attached to the support. In this reaction scheme, the stepwise addition of individual monomers to a single, growing end of a oligonucleotide ensures accurate synthesis of the desired sequence. Moreover, unwanted side reactions are eliminated, such as the condensation of two oligonucleotides, resulting in high product yields.

In addition, oligonucleotides can be synthesized from nucleotide triplets. Here, a triplet coding for each of the twenty amino acids is synthesized from individual monomers. Once synthesized, the triplets are used in the coupling reactions instead of individual monomers. However, the cost of synthesis from such triplets far exceeds that of synthesis from individual monomers because triplets are not commercially available.

Oligonucleotide synthesis on a solid support has also been adapted to produce a desired sequence of duplex DNA, e.g., a particular gene (see, e.g., reviewed, e.g. in Beattie & Fowler, Nature 352:548-549, 1991). For example, solid-phase gene assembly techniques have been described in which an oligonucleotide bound to a support is annealed to the next oligonucleotide encoding the desired region of a gene. After washing away unbound oligonucleotides, repeated steps of hybridization and washing are performed to assemble the particular gene of interest. The segments can be ligated and then the sequence is removed from the solid support and ligated into a vector. Such techniques have been used to assemble various genes (see, e.g., Stahl et al., Biotechniques 14:424-434, 1993; Hostomsky & Smrt, Nucleic Acids Symp Ser 18:241-244, 1987; Hostomsky, et. al, Nucleic Acids Res 15:4849-56, 1987).

Although, the solid phase gene assembly technique can provide a particular product, e.g., a desired gene it requires synthesis of a large number of oligonucleotides in order to assemble the gene. While some diversity may be introduced into the sequence, e.g. random nucleotides positioned at particular regions in one or more of the oligonucleotides, the technique does not lend itself to generating libraries of double-stranded sequences that contain blocks of diverse, i.e., different, segments as the number of oligonucletides required to provide diversity would be very large.

In vivo recombination can be utilized to produce new polynucleotides from component genetic elements. Cells with high levels of homologous recombination activity can be used to recombine various genetic elements. In one method, termed “exon shuffling”, genetic elements are flanked by homologous sequences within introns then transferred to host cells that produce recombination between the homologous segments (Kolkman, J. A. and W. P. Stemmer (2001). Nat. Biotechnol. 19: 423-428). A population of polynucleotides is produced with different combinations of exon segments and introns.

Traditional methods to synthesize genetic constructs rely on cloning techniques performed in solution. While these methods can be robust, they suffer serious limitations when more than three genetic elements are to be coupled to one another in an ordered manner. For example, if one wants to couple four genetic elements together, there are 256 (4⁴) possible different tetrameric molecules that would result if the coupling process is random. The randomness may be altered by engineering restriction sites, or overhangs, at the ends of the molecules to be coupled such that DNA basepairing favors certain genetic elements to be coupled to ends with the appropriate complement. This process is often tedious, and may not be amenable to certain genetic elements.

If several modules of genetic material were to be coupled in a desired order, they would have to be cloned in a stepwise fashion, which would take several days. Furthermore, if multiple homologs of the modules are to be coupled at each step, the number of resulting different clones increase exponentially with the number of couplings and linearly with the number of domains to be coupled. Generally this can be expressed as N=M^{C} where N is the total number of different sequences produced, M is the number of modules coupled at each step, and C is the number of coupling steps. Thus, for a molecule that has 3 homologs to be coupled at each step, 3 coupling events would produce 3³=27 different molecules. Standard solution-based molecular biology approaches would take days if not weeks to produce this many clones, whereas solid-phase based cloning can produce them in a single day.

Thus, although cloning and recombinant DNA techniques have been practiced for several years, there are shortcomings to the common techniques. First, blunt ended ligation proceeds at a much lower rate than ligation with ends containing overhangs. Second, orientation of the genetic elements being coupled is not easily well controlled without significant engineering of restriction sites into the fragments. Third, concatemers of polynucleotides form easily in solution reactions, such that the desired polynucleotide to be produced is often not efficiently generated. Fourth, cloning of multiple segments in a desired order has been virtually impossible due to the exponential increase in irrelevant couplings when ligation occurs in solution. Because of the aforementioned limitations, cloning of more than two or three genetic elements in a desired order cannot be produced efficiently and on a large scale.

### Molecular Evolution

Molecular evolution technologies have relied on the production of novel gene sequences using technologies based on sequence homology. DNA shuffling is a method that produces hybrid genes from homologous starting sequences. Because the method relies on PCR, the diversity of the libraries produced by this method is limited by homology limitations inherent in PCR. In nature, several functionally and structurally similar proteins have been produced through evolution that do not share highly homologous DNA sequences at the genetic level. Thus, molecular evolution technologies could benefit from methods that can produce novel sequences without the inherent limitations of PCR based shuffling protocols. The ability to create novel libraries of polynucleotides from starting sequences showing low homology would powerfully affect molecular evolution techniques.

In recent years, detailed structural information has been elucidated for several proteins in nature through protein crystallography (www.rscb.org/pdb). Protein structures can be classified into different “folds” based on the three dimensional structure. Comparison of the structures has revealed that the number of different folds in all of nature is likely limited to around 1000 different folds (Domingues, F. S., W. A. Koppensteiner, et al. (2000). FEBS Letters 476: 98-102.; Gerstein, M. (2000). Nat. Struct. Biol. structural genomics supplement: 960-963.). Folds are often not homologous at the DNA or amino acid level, but share homology in three dimensional space based on the conformation of the α-carbon chain and of interacting amino acids. Thus, all biochemical processes are carried out by a limited set of general fold structures. The implication of these observations for evolutionary processes is that novel enzymes should be comprised of amino acid sequences that form conformations that fit into one of the known fold structures. Engineering technologies also should allow folds to be produced that are found in nature. Although homology-based methods can produce novel sequences encoding proteins conforming to specific folds, most of the sequence space comprising known folds is unattainable through homology constrained processes. The ability to harness various amino acid sequences already found in nature through non-homologous means to produce novel proteins that conform to the known fold structures would be useful in protein engineering.

Non-homologous processes occur in nature to produce novel gene sequences. Several gene families are thought to have arisen through “gene swapping” events. For example, polyketide synthetase pathways are formed by genes arranged in a modular fashion, with various modules encoding different enzyme functions (Tsuji, S. Y., N. Wu, et al. (2001). Biochemistry 40: 2317-2325). Several proteins form the mammalian clotting cascade have evolved through non-homologous mechanisms. Various members of the clotting pathway are comprised of modules consisting of protein targeting domains fused with protease activities. Several members of the splicing machinery also are composed of varying domain structures.

### Antibody Discovery

Additionally, antibody genes are organized in a segmental fashion in the genome, with functional genes being created by non-homologous recombinational events. Current methods do not allow the de novo creation of functional antibody genes in vitro, a process which would be an efficient way to discover and produce this important class of human therapeutics.

Antibody diversity is harnessed in the pharmaceutical industry by utilizing antibody therapeutics. In this technology, an antibody must be discovered which binds to an important therapeutic target. This target may be a protein in the serum, a cell surface protein, a molecule on or within a cell, or a molecule comprising a pathogenic organism like a bacteria or virus. Antibodies are also utilized as diagnostic reagents or as tools in molecular biology research. Thus, the ability to produce and identify antibodies is of significant medical, industrial, and economical importance.

Classic techniques to produce antibodies include hybridoma technologies, wherein a mouse is immunized with an antigen and cells secreting a specific antibody are fused to immortalized cells such that antibody secreting cells may be propagated in the laboratory. This technique is time consuming, costly, and often not effective for generating high affinity antibodies. Further, classic technologies produce mouse antibodies which are not as useful therapeutically as human antibodies due to cross-species immunogenicity. More recent advances make use of transgenic mice which have replaced the endogenous murine antibody locus with the human antibody locus. These mice produce fully human antibodies, but suffer from the selection mechanisms that delete self reactive antibodies, which could be useful as therapeutics.

Other more recent methods for generating antibodies in vitro have been described. These generally rely on the isolation of antibody cDNA from B-cells and expression of the encoded antibody fragment on the surface of a phage. In such “phage display” experiments an antibody fragment that binds a substrate is identified and the gene encoding it obtained by isolating the relevant phage by panning techniques. Such methods can identify fragments of antibodies that bind certain antigens but suffer on several fronts: antibody fragments are often not as useful clinically due to poor pharmacokinetics and biodistribution, a full antibody molecule might contain higher affinity properties due to the association of light and heavy chains in the proper folding confirmation, cDNA libraries contain DNA that has been selected in vivo and thus might not contain relevant sequences with high binding affinity for certain antigens (such as self antigens).

Thus, an in vitro method to produce antibodies from the genetic elements from which they are naturally comprised would provide a robust method for both synthesis as well as discovery of novel antibodies.

### BRIEF SUMMARY OF THE INVENTION

The present invention provides methods to combinatorially arrange genetic elements such that novel polynucleotides are produced. Specifically, it provides a method whereby a genetic element is immobilized to a solid support and a second genetic element is covalently attached to said first immobilized genetic element, such that a new genetic element is formed which comprises the first and second genetic element. This process may be repeated several times to produce polynucleotides comprising several genetic elements linked in an ordered fashion as determined by the researcher. Also, the invention provides a means to cleave the polynucleotide from the solid support, such that it can be used as a vector in solution. Following cleavage the polynucleotides may optionally be ligated to form circular polynucleotides, such as a plasmid vector. Further, the invention provides a population of polynucleotides, differing from one another by modular genetic sequences produced as a result of combinatorial synthesis of genetic elements on a solid support.

The present invention is directed towards novel methods for assembly and detection of a polynucleotide on a solid-support. The methods are directed to rapid, efficient, low-cost, and large-scale synthesis of polynucleotides for use, for example, as synthetic genes for recombinant protein expression, as vectors for gene expression, as libraries for molecular evolution purposes, as therapeutic agents, and as probes for diagnostic assays. The resulting polynucleotides on a solid-support can be (i) amplified by the polymerase chain reaction (PCR), (ii) manipulated for useful purposes while attached to the solid-support, (iii) quantitated and detected by fluorescence-based, hybridization and exonuclease assays, (iv) expressed directly from the solid support by in vitro transcription or translation, (v) cleaved from the solid-support to produce polynucleotides in solution, or (vi) selected or screened for altered or enhanced function.

Certain aspects and embodiments of the present invention obviate many of the limitations and imperfections of the classic method of gene synthesis and confer some or all of the following advantages:

1) The solid support serves to allow the genetic elements to be coupled to one another in an ordered fashion, such that the resulting polynucleotide comprises the modular genetic elements linked to one another in an order determined a priori.

2) The solid support serves to allow efficient washing and removal of excess and non-ligated polynucleotides, by-products, reagents, and contaminants. Purifications prior to the completion of gene assembly are not necessary.

3) The solid support eliminates the problem of concatemers forming in solution during ligation steps, and contaminating the final product.

4) Libraries of polynucleotides can be produced which do not rely on homologous sequences of the starting material, but which may encode similar protein secondary structures, a feature important in molecular evolution technologies.

5) Libraries of polynucleotides containing combinatorial deletions or additions of modular genetic elements can be produced by varying the coupling efficiency at each step.

6) The modular genetic elements can be relatively short, therefore they will be inexpensive, highly pure, and readily available. Also, modular genetic elements can be synthesized by PCR. Further, long genetic elements can also be utilized, such as linearized plasmid DNA.

7) Further experiments can be conducted on the assembled polynucleotide while immobilized on the solid-support, such as transcription, translation, or nucleic acid/protein binding assays.

In particular, the invention provides a method of preparing a library of double-stranded polynucleotides, each of which encodes a multiplicity of genetic elements, the method comprising: providing a first population of double-stranded polynucleotides, wherein the first population is immobilized to a solid support in a non-addressable configuration; providing a second population of double-stranded polynucleotides, wherein the second population comprises a multiplicity of different sequences encoding a genetic element; and covalently coupling the second population to the first population to create a library of double-stranded polynucleotides in a non-addressable configuration.

In some embodiments, the first population of polynucleotides may also comprise a multiplicity of different sequences encoding a genetic element, often a polypeptide domain. In one embodiment, the polypeptide domain encoded by the genetic element is an antibody segment selected from the group consisting of a V, D, or J segment. For example, the antibody segment may be a V segment. In other embodiments, the polypeptide domain may be a kringle domain, a fragment of carbon-carbon lyases, or a domain selected from the group consisting of α-helices, β-strands, β-sheets, β-turns, and loops. The second population of polynucleotides encoding a genetic element, which population is joined to the first, may encode another polypeptide domain. For example, the second population may encode an antibody J segment, which is then linked to the V segment encoded by the first population.

Often, the invention further comprises a step of covalently coupling a third population of double-stranded polynucleotides to the immobilized sequence that comprising the first and second populations. The third population may also comprise a multiplicity of different double-stranded polynucleotides encoding a genetic element.

In some embodiments of the method, the second polynucleotide population is blocked, often by dephosphorylating the ends of the double-stranded oligonucletodie with a phosphatase. The method may comprise an addition step of deblocking the immobilized polynucleotides, for example, by phosphorylating the 5′end.

In additional embodiments, the method comprises a step of transcribing and translating the immobilized polynucleotide. a step of cleaving the immobilized polynucleotides from the solid support.

The invention also provides a method further comprising a step of cleaving the resulting population of double-stranded nucleic acid from the solid support. The cleaved double-stranded nucleic acid library may be ligated to produce circular polynucleotides.

The methods of the invention may also comprise a step of screening the library to identify a member that has a particular desired function, e.g., binding to a particular antigen.

The invention also provides libraries prepared using the methods described herein. The library may be immobilized in a non-addressable configuration.

### BRIEF DESCRIPTION OF THE DRAWINGS

FIG. 1. Illustrates a scheme for construction of a polynucleotide on a solid support. The black rectangle represents a solid support to which a nucleic acid is immobilized. An incoming nucleic acid is then coupled by a ligase to the immobilized nucleic acid. This process can be repeated a multitude of times. In this figure, blocking is illustrated by the absence of a phosphate (represented by a “P” in a circle), and deblocking by the addition of a phosphate. Addition of a phosphate may be accomplished by a kinase.

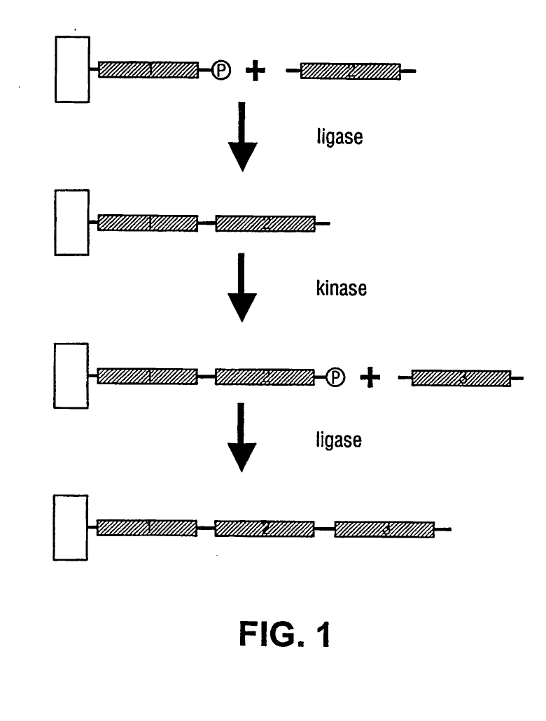

FIG. 2. Shows the immobilization of a nucleic acid to a solid support. A streptavidin plate (Pierce, Rockford, Ill.) was either exposed to buffer (first bar), or 200 ng of the biotinylated 32 basepair double-stranded oligonucleotide f32 (second bar), followed by washing the plate three times in buffer, then staining with the DNA specific dye picogreen (Molecular Probes, Eugene, Oreg.).

FIG. 3. Shows the coupling of an incoming polynucleotide to an immobilized oligonucleotide. Wells of a streptavidin coated plate were either exposed to buffer (bar 1), or biotinylated f32 (bars 3-5) followed by washing the wells three times with buffer. Bars 3 and 4 were then exposed to pBluescript plasmid linearized with Sma I, either in the absence (bar 3) or presence (bar 4) of T4 DNA ligase. Wells were stained with picogreen to detect the presence of DNA.

FIG. 4. Shows that immobilized polynucleotides can be cleaved from a solid support. In the presence of the restriction enzyme Pvu II or Xba I, the staining of immobilized nucleic acids decreases. The graphic below the bar graph shows the location of restriction sites on the immobilized DNA comprised of biotinylated f32 ligated to linear pBluescript.

FIG. 5. Shows a method for blocking and deblocking incoming or immobilized nucleic acids. Various combinations of phosphorylated f32, dephosphorylated f32 (by CIP treatment), phosphorylated pBluescript/Sma I, or dephosphorylated pBluescript/Sma I (by CIP treatment) were tested in coupling reactions with T4 DNA ligase. The extent of ligation was monitored by picogreen staining of the streptavidin coated wells of a microtiter plate.

FIG. 6 shows that a blocked immobilized polynucleotide can be deblocked by the addition of a phosphate by polynucleotide kinase. The deblocked oligonucleotide is then available to couple to an incoming polynucleotide.

### DETAILED DESCRIPTION OF THE INVENTION

### Introduction

Methods to synthesize genetic constructs rely on cloning techniques performed in solution. While these methods can be robust, they suffer serious limitations when more than three genetic elements are to be coupled to one another in an ordered manner. For example, if one wants to couple four genetic elements together, there are 256 (4⁴) possible different tetrameric molecules that would result if the coupling process is random. The randomness may be altered by engineering restriction sites, or overhangs, at the ends of the molecules to be coupled such that DNA basepairing favors certain genetic elements to be coupled to ends with the appropriate complement. This process is often tedious, and may not be amenable to certain genetic elements. The present invention provides a means to couple genetic elements on a solid support, such that a first polynucleotide is immobilized on a solid support and a second polynucleotide is coupled to the first. Following coupling, the components of the coupling reaction are washed away, leaving a new polynucleotide comprising the first and second genetic elements coupled to the solid support. This process can be continued such that a third polynucleotide is then coupled to the immobilized polynucleotide. Further, the process may be repeated a plurality of times. This process eliminates the irrelevant molecules produced from solution-based cloning approaches and allows efficient construction of any modular polynucleotide desired.

The present invention has important applications in several areas of biotechnology:

1) For recombinant DNA techniques, solid-support mediated cloning allows for the directional cloning of several DNA segments in an ordered fashion and in a high-throughput manner,

2) For molecular evolution, solid-support mediated cloning allows for the development of large libraries of polynucleotides formed through non-homologous means,

3) For fusion molecule synthesis, solid-support mediated cloning allows for the production of novel gene fusions in a rapid, ordered, and high-throughput manner, and

4) In de novo antibody gene synthesis, solid-support mediated cloning allows for the production of full length antibody genes from their component V, D, J or C gene segments in vitro.

As appreciated by one of skill in the art, multiple libraries created using the methods of the invention may also be created in parallel. For example mulitiple libraries may be constructed on a microtiter plate or an array. Each well of the microtiter plate or spot of the array would constitute a distinct library as defined herein.

The following definitions will be useful in understanding the present invention.

### Definitions

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

“Polypeptide” and “peptide” are used interchangeably herein to refer to a polymer of amino acid residues; whereas a “protein” typically contains one or multiple polypeptide chains. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

In the polypeptide notation used herein, the left-hand direction is the amino terminal direction and the right-hand direction is the carboxy-terminal direction, in accordance with standard usage and convention. Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the coding RNA transcript are referred to as “downstream sequences”.

“Domain” refers to a unit of a protein or protein complex, comprising a polypeptide subsequence, a complete polypeptide sequence, or a plurality of polypeptide sequences where that unit has a defined function. The function is understood to be broadly defined and can be ligand binding, catalytic activity or can have a stabilizing effect on the structure of the protein.

An “antibody” refers to a protein of the immunoglobulin family or a polypeptide comprising fragments of an immunoglobulin that is capable of noncovalently, reversibly, and in a specific manner binding a corresponding antigen. An exemplary antibody structural unit comprises a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains respectively.

Antibody genes are comprised of gene segments. These segments are given the terms variable (abbreviated “V”), diversity (abbreviated “D”), junctional (abbreviated “J”), and constant (abbreviated “C”). There are two polypeptides of which an antibody is comprised: a light chain and a heavy chain. The polypeptide of a heavy chain is encoded by DNA comprised of V, D, J, and C genetic elements. A light chain polypeptide is encoded by DNA comprised of V, J, and C genetic elements. There are several V, D, and J segments comprising the antibody locus in the germline from which a rearranged functional gene may be derived. The sequences of V, D, and J segments are known and are available in public databases.

The term “function of interest” refers to any phenotypic change induced by a genetic alteration without limitation. It also includes in vitro changes to proteins such as improved enzymes. More specifically, the function of interest relates to a biological activity and can be the loss or gain or improvement of enzymatic (activity) function, resistance to selective pressure such as environmental toxicity, improved resistance to pathogens, alterations in cell development, alterations in tumorigenicity, alterations in cell invasiveness as in metastatic cancers, protein stability and protein binding (affinity constants, i.e. antibodies or ligand binding).

The term “residue” as it relates to a polynucleotide or polypeptide refers to either a purine or pyrimidine nucleotide for polynucleotides, or an amino acid for a polypeptide.

A “genetic element” means a sequence of polynucleotides encoding a function. For example, a “genetic element” may encode a polypeptide sequence, may encode a promoter function, an enhancer function, a transcription start or stop site, or RNA splice sites and the like. A “genetic element” may also refer to particular structural feature, e.g., an intron, or a polyA tail. Genetic elements may be operatively linked to other genetic elements, for example a promoter may be operatively linked to a genetic element encoding a protein to allow expression of a protein in a given cell type. A sequence encoding a genetic element may also comprise additional sequences that do not encode the particular function.

The terms “gene” and “gene of interest” refer to a polynucleotide that encodes a polypeptide.

The term “swap” or “gene swapping” in reference to a polynucleotide means either: 1) the occurrence of a deletion of at least two residues occupying consecutive positions in a polynucleotide, or 2) the occurrence of an addition of at least two residues occupying consecutive positions into a polynucleotide, or 3) the replacement of at least two residues occupying consecutive positions in a polynucleotide with other residues.

The term “library of polynucleotide sequences” refers to a mixture of polynucleotides, wherein at least one of the sequences differs from at least one other sequence in the mixture by sequence composition or length, for example, where at least one position is occupied by a different nucleotide when the two sequences are compared or at least one nucleotide position is absent in one sequence when compared with the other sequence.

“Diverse” as used herein refers to a population of nucleic acid molecules, that have at least two sequences that are different in composition or length.

The term “DNA” refers to deoxyribonucleic acid. It will be understood by those of skill in the art that where manipulations are described herein that relate to DNA they will also apply to RNA.

The term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later. Preferably the region of identity is greater than about 5 bp, more preferably the region of identity is greater than 10 bp. Thus, “homologs” are nucleic acid molecules that are not identical but are capable of hybridizing to one another under physiological conditions. Double-stranded homologs are capable of hybridizing to one another following denaturation.

The term “heterologous” as used herein in the context of a chimeric polynucleotide or, refers to sequences comprising segments, domains, or genetic elements, the exact combination and sequence of which is not found in nature.

The term “identical” or “identity” means that two nucleic acid sequences or polypeptide seqeucnes have the same sequence. Thus, “areas of identity” means that regions or areas of a nucleic acid fragment or polynucleotide are identical to another polynucleotide or nucleic acid fragment.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

The term “amplification” means that the number of copies of a nucleic acid fragment is increased.

The term “wild-type” means that the nucleic acid fragment does not comprise any mutations. A “wild-type” protein means that the protein will be active at a comparable level of activity found in nature and typically will comprise the amino acid sequence found in nature. In an aspect of the invention, the term “wild type” or “parental sequence” can indicate a starting or reference sequence prior to a manipulation of the sequence.

The term “related polynucleotides” means that regions or areas of the polynucleotides are identical and regions or areas of the polynucleotides are heterologous.

The term “chimeric polynucleotide” refers to a polynucleotide that comprises wild-type sequences and sequences that are mutated. It also refers to a polynucleotide comprising heterologous segments of polynucleotides.

The term “population” as used herein means a collection of components such as polynucleotides, nucleic acid fragments or proteins. A “mixed population” means a collection of components which belong to the same family of nucleic acids or proteins (i.e. are related) but which differ in their sequence (i.e. are not identical) and hence in their biological activity. A “library” necessarily implies a population wherein at least two of the components are different in some aspect (chemical composition, length, etc.).

The term “first population” of nucleic acids does not require that such a population be directly linked to the solid support. The first population may be immobilized to the solid support via another nucleic acid sequence.

The term “specific nucleic acid fragment” means a nucleic acid fragment having certain end points and having a certain nucleic acid sequence. Two nucleic acid fragments wherein one nucleic acid fragment has the identical sequence as a portion of the second nucleic acid fragment but different ends comprise two different specific nucleic acid fragments. Two nucleic acid fragments with identical sequences but different 5′ or 3′ ends comprise two different specific nucleic acid fragments.

The term “mutations” means changes in the sequence of a wild-type nucleic acid sequence or changes in the sequence of a peptide. Such mutations may be point mutations such as transitions or transversions. The mutations may be deletions, insertions or duplications.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. Generally, the term naturally-occurring refers to an object as present in a non-pathological (undiseased) individual, such as would be typical for the species.

As used herein the term “physiological conditions” refers to temperature, pH, ionic strength, viscosity, and like biochemical parameters which are compatible with a viable organism, and/or which typically exist intracellularly in a viable cultured yeast cell or mammalian cell. For example, the intracellular conditions in a yeast cell grown under typical laboratory culture conditions are physiological conditions. Suitable in vitro reaction conditions for in vitro transcription cocktails are generally physiological conditions. In general, in vitro physiological conditions comprise 50-200 mM NaCl or KCl, pH 6.5-8.5, 20-45° C. and 0.001-10 mM divalent cation (e.g., Mg⁺⁺, Ca⁺⁺); preferably about 150 mM NaCl or KCl, pH 7.2-7.6, 5 mM divalent cation, and often include 0.01-1.0 percent nonspecific protein (e.g., BSA). A non-ionic detergent (Tween, NP-40, Triton X-100) can often be present, usually at about 0.001 to 2%, typically 0.05-0.2% (v/v). Particular aqueous conditions may be selected by the practitioner according to conventional methods. For general guidance, the following buffered aqueous conditions may be applicable: 10-250 mM NaCl, 5-50 mM Tris HCl, pH 5-8, with optional addition of divalent cation(s) and/or metal chelators and/or nonionic detergents and/or membrane fractions and/or antifoam agents and/or scintillants.

As used herein, “a peptide linker” or “spacer” refers to a molecule or group of molecules that connects two molecules, such as a DNA binding protein and a random peptide, and serves to place the two molecules in a preferred configuration, e.g., so that the random peptide can bind to a receptor with minimal steric hindrance from the DNA binding protein. Tow molecule can be linked chemically or by recombinant means. Sequence encoding peptide linker or spacer molecules, e.g., Gly, Ser, Ala linkers, may also be introduced into the double-stranded nucleic acid synthesized on the solid support to link particular domains or fragments in a desired configuration.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

“Attachment site” refers to the atom on an oligonucleotide to which is attached a linker.

“Linker” refers to one or more atoms connecting an oligonucleotide to a solid-support, label, or other moiety.

The term “solid-support” refers to a material in the solid-phase that interacts with reagents in the liquid phase by heterogeneous reactions. Solid-supports can be derivatized with oligonucleotides by covalent or non-covalent bonding through one or more attachment sites, thereby “immobilizing” an oligonucleotide to the solid-support.

A “non-addressable configuration” as used herein refers to immobilization of a population of nucleic acids to a solid support such that the nucleic acid molecules are not in a predetermined location. Typically, each immobilized nucleic acid has an equal probability of having any of the incoming polynucleotides in a population joined to it.

The term “incoming polynucleotides” as used herein refers to a population of double-stranded nucleic acids that are contacted with a population of immobilized polynucleotides and coupled to the immobilized polynucletoides thereby creating a population of immobilized polynucleotides comprising the incoming sequences. The “incoming polynucleotides” may comprise sequence encoding polypeptides, poypeptide domain, genetic elements, e.g., promoters, enhancers, as well as other sequences, e.g., linker sequences. The incoming polynucleotides typically encode a multiplicity of sequences.

The term “overhang” refers to a single-stranded terminus of a duplex of base-paired oligonucleotides. The overhang may be one or more bases in length and allows for annealing of a complementary oligonucleotide prior to ligation and extension during polynucleotide assembly.

The term “ligate” refers to the reaction of covalently joining adjacent oligonucleotides through formation of an internucleotide linkage.

The term “ligase” refers to a class of enzymes and their functions in forming a phosphodiester bond in adjacent oligonucleotides which are annealed to the same oligonucleotide. Particularly efficient ligation takes place when the terminal phosphate of one oligonucleotide and the terminal hydroxyl group of an adjacent second oligonucleotide are annealed together across from their complementary sequences within a double helix, i.e. where the ligation process ligates a “nick” at a ligatable nick site and creates a complementary duplex (Blackburn, M. and Gait, M. (1996) in Nucleic Acids in Chemistry and Biology, Oxford University Press, Oxford, pp. 132-33, 481-2). The site between the adjacent oligonucleotides is referred to as the “ligatable nick site”, “nick site”, or “nick”, whereby the phosphodiester bond is non-existent, or cleaved.

The term “DNA ends” or “ends” refers to the position in a DNA strand wherein a phosphodiester bond is broken. In a single-stranded DNA end a nucleotide is only covalently linked with one other nucleotide. A “double-stranded DNA or RNA end” refers to the position in a double-stranded DNA molecule wherein the molecule is no longer double-stranded. Generally DNA ends are recognizable to those skilled in the art. Double-stranded DNA ends are characterized as blunt, having a 5′ overhang, a 3′ overhang, or a hairpin structure. A DNA end may or may not contain a 5′ phosphate group.

The term “cleavage” as used herein refers to the breakage of a bond between two nucleotides, such as a phosphodiester bond.

The term “circular polynucleotide” refers to a polynucleotide wherein no double-stranded DNA ends are present if the polynucleotide is double stranded, or no single-stranded DNA ends are present if the molecule is single-stranded. A circular polynucleotide may be single-stranded or double-stranded. A circular polynucleotide may, however, contain single-stranded DNA ends if the molecule is double stranded. A circular polynucleotide will be present if single-stranded DNA ends exist but hydrogen bonding keeps the two strands of the double-stranded molecule hybridized to one another such that a double-stranded DNA end is not created by the presence of two single-stranded ends in proximity to one another. Such a circular double-stranded polynucleotide is often referred to as “nicked”.

The term “linear polynucleotide” is a polynucleotide which contains at least one, but most often two DNA ends. A linear polynucleotide may be either single-stranded or double-stranded.

As used herein, “substantially pure” means an object species is the predominant species present (i.e., on a molar basis it is more abundant than any other individual macromolecular species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. Solvent species, small molecules (<500 Daltons), and elemental ion species are not considered macromolecular species.

“Kringle domains” are autonomous structural domains, found throughout the blood clotting and fibrinolytic proteins. Kringle domains are believed to play a role in binding mediators (e.g., membranes, other proteins or phospholipids), and in the regulation of proteolytic activity. Kringle domains are characterized by a triple loop, 3-disulphide bridge structure, whose conformation is defined by a number of hydrogen bonds and small pieces of anti-parallel beta-sheet. They are typically between 70 and 90 amino acids long. Plasminogen-like kringles possess affinity for free lysine and lysine-containing peptides. They are found in a varying number of copies (up to 38 in apolipoprotein(a)) in some serine proteases and plasma proteins.

### Generation of Double-stranded Polynucletide Libraries

This invention relies on routine techniques in the field of recombinant genetics. Basic texts disclosing the general methods of use in this invention include Sambrook and Russell, Molecular Cloning: A Laboratory Manual 3d ed. (2001); Kriegler, Gene Transfer and Expression: A Laboratory Manual (1990); and Ausubel et al., Current Protocols in Molecular Biology (1994).

For nucleic acids, sizes are given in either kilobases (Kb) or base pairs (bp). These are estimates derived from agarose or polyacrylamide gel electrophoresis, from sequenced nucleic acids, or from published DNA sequences. For proteins, sizes are given in kilo-Daltons (kD) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage and Caruthers, Tetrahedron Letters, 22:1859-1862 (1981), using an automated synthesizer, as described in Van Devanter et al., Nucleic Acids Res., 12:6159-6168 (1984). Purification of oligonucleotides is by either native polyacrylamide gel electrophoresis or by anion-exchange chromatography as described in Pearson & Reanier, J. Chrom., 255:137-149 (1983). The sequence of the cloned genes and synthetic oligonucleotides can be verified after cloning using, e.g., the chain termination method for sequencing double-stranded templates of Wallace et al., Gene, 16:21-26 (1981).

### Immobilized and Incoming Polynucleotide Sequences

The present invention requires (i) polynucleotide(s) of interest to be coupled, (ii) a solid support material, and (iii) a means to couple said polynucleotides. A means to prevent (block) unwanted couplings from occurring is also preferred. Optionally, the invention could utilize a means of cleaving the polynucleotide from the solid support, and a means to circularize the cleaved polynucleotides.

The present invention can be applied to produce any polynucleotide of interest to the researcher. The polynucleotide can be nucleic acid, i.e. RNA or DNA. Often the polynucleotide will be DNA consisting of genetic elements or one or more genes of interest. The polynucleotide to be produced will preferentially be double-stranded and may be comprised of domains, or modules. The polynucleotide to be produced may be a gene, a domain, a combination of genetic elements, a regulatory sequence, or a vector comprising a plurality of genetic elements.

In order to produce the resulting polynucleotide, the genetic elements of which the polynucleotide is to be comprised must be obtained as starting material. The starting material may be obtained through natural sources, or may be polynucleotides which have been synthesized in a laboratory (e.g. gene synthesis), or may be polynucleotides derived from natural sources which have been manipulated in a laboratory. Polynucleotide sequence of various genes or gene segments of interest, e.g., V, D, J segments of antibody genes, are available through publicly held databanks such as Genbank or available commercially (Celera, Rockville, Md.; Incyte, Palo Alto, Calif.; Clontech, Palo Alto, Calif.; Invitrogen, Carlsbad, Calif.).

Starting nucleic acid may be obtained from cloned DNA or RNA or from natural DNA or RNA from any source including bacteria, yeast, viruses, plants, animals. Fragments may be directly obtained, for example, by screening libraries for the desired sequences and subcloning the sequences into a vector to produce large quantitites, or may be obtained through amplification methods such as the polymerase chain reaction (PCR) using a nucleic acid template, e.g., genomic DNA, cDNA, RNA, or other recombinant DNA, to obtain the sequence of interest.

Alternatively, the polynucleotide may be present as a cloned sequence in a vector and sufficient nucleic acid may be obtained.

The choice of vector depends on the size of the polynucleotide sequence and the host cell to be employed in the methods of this invention. The templates may be plasmids, phages, cosmids, phagemids, viruses (e.g., retroviruses, parainfluenzavirus, herpesviruses, reoviruses, paramyxoviruses, and the like), or selected portions thereof (e.g., coat protein, spike glycoprotein, capsid protein). For example, cosmids, phagemids, YACs, and BACs are preferred where the specific nucleic acid sequence to be mutated is larger because these vectors are able to stably propagate large nucleic acid fragments.

If the specific nucleic acid sequence is cloned into a vector it can be clonally amplified by inserting each vector into a host cell and allowing the host cell to amplify the vector. This is referred to as clonal amplification because while the absolute number of nucleic acid sequences increases, the number of mutants does not increase.

The starting DNA to be immobilized may be single-stranded or double stranded, Preferentially, the length of the nucleic acid to be immobilized will be more than four nucleotides, and is preferentially more than fifteen nucleotides. Optionally, the length can be greater than 100 or 1000 nucleotides. The “incoming” polynucleotide to be coupled is preferentially greater than four nucleotides, and is optionally more than 100 or 1000 nucleotides.

Starting material may be blunt-ended or contain overhangs. Often it is desirable to ligate the polynucleotide in a certain orientation. In such cases it is preferred that at least one of the DNA ends of the incoming polynucleotide contains an overhang. The overhang can be of either the 3′ of 5′ end, and may be of any length. Overhangs can be produced by restriction enzymes acting at recognition sites at the end of the polynucleotide to be coupled. Some restriction enzymes can recognize a specific sequence, yet cleave DNA at a site that is independent of sequence, and is distant from the recognition site. These enzymes will allow overhangs to be produced without the need to engineer recognition sites that would be present in the final product. Alternatively, overhangs can be produced without the use of restriction enzymes. The enzyme terminal deoxynucleotidyl transferase (TdT) will add nucleotides to the 3′ end of a DNA molecule, thus producing an overhang. Other polymerases, like Taq polymerase, is capable of adding adenines to the ends of DNA to produce an overhang. If directionality is desired in the ligation of the incoming polynucleotide, it is preferable that the overhangs of the two ends comprise different sequences, or that one end contains an overhang while the other end is blunt.

The immobilized DNA or the incoming genetic elements may be DNA cleaved at a random position by an enzyme such as S1 nuclease as described in WO02/16642. This DNA cleaved at a random position can be coupled to the solid support such that further coupling events insert genetic elements at random positions in the growing polynucleotide chain.

Starting material should be in substantially pure form. The polynucleotide may be double-stranded or single-stranded, but more preferably is double-stranded. Further, the polynucleotide may be linear or circular, but in a preferred embodiment the polynucleotide is linear. Polynucleotides in linear form may be prepared by techniques well known to those skilled in the art (see, e.g., Sambrook and Russell, supra). The number of different specific nucleic acid fragments in the reaction vessel will be at least about 10, preferably at least about 50, and more preferably at least about 100.

The polynucleotides may comprise a number of different segments or elements, include both coding and non-coding regions. For example, a number of genetic elements, e.g. promoters, enhancer, can be used. Other elements include insulators, which are genetic elements generally found in eukaryotic cells that affect chromatin structure, and allow differential regulation of adjacent genes (Bell, et al. Science (2001) 291: 447-450). Introns can also be included, e.g., for enhancing gene expression through the coupling of transcription to translation (Berget, S. M. J. Biol. Chem. (1995) 270: 2411-2414). Intron enhancers may enhance the recognition of splice signals by the spliceosome. Poly A tails may also be included. Protein coding regions are nucleic acid sequences that specify, or encode, a particular polypeptide sequence. Any of the above, or other genetic elements could feasibly be used in the present invention.

In generating the sequences of the invention, at least one population of incoming polynucleotides at a stage of assembly of the library of sequences comprises a mixture of nucleic acids such that diversity is created in the population of molecules created on the solid support. Diversity can be created by: 1) the use of homologs at each coupling step, 2) the use of nucleic acids encoding similar domain structures, 3) the use of random nucleic acid fragments, or 4) the use of a mutagenesis procedure (like error-prone PCR, passage through mutator strains or DNA shuffling) to generate diversity in a parent polynucleotide.

### Solid Supports

Oligonucleotides may be immobilized on solid supports through any one of a variety of well-known covalent linkages or non-covalent interactions. The support is comprised of insoluble materials, preferably having a rigid or semi-rigid character, and may be any shape, e.g. spherical, as in beads, rectangular, irregular particles, resins, gels, microspheres, or substantially flat as in a microchip. In some embodiments, it may be desirable to create an array of physically separate synthesis regions on the support with, for example, wells, raised regions, dimples, pins, trenches, rods, pins, inner or outer walls of cylinders, and the like.

Preferred support materials include agarose, polyacrylamide, magnetic beads (Stamm, S. and Brosius, J. (1995) “Solid phase PCR” in PCR 2, A Practical Approach, IRL Press at Oxford University Press, Oxford, U.K., p. 55-70.), polystyrene (Andrus, et. al. Nucleic Acids Symp Ser. 1993; 29:5-6.), controlled-pore-glass (Caruthers, Science (1985) 230: 281-5.), polyacrylate hydroxethylmethacrylate, polyamide, polyethylene, polyethyleneoxy, or copolymers and grafts of such. The hydrophilic nature of the polyethyleneoxy groups promotes rapid kinetics and binding when aqueous solvents are used. If magnetic beads are used, they should be encapsulated in a substance that allows them to be compatible with enzymatic systems. Polystyrene coated paramagnetic particles are commercially available (Bangs Laboratories, Fishers, Ind.) and are suitable in practicing the present invention.

Other solid-supports include small particles, membranes, frits, non-porous surfaces, addressable arrays, vectors, plasmids, or polynucleotide-immobilizing media. Additionally, fullerenes can conceivably be used as a solid support, as well as derivatized fullerenes such as gadolinium fullerenes which contain paramagnetic properties.

Immobilization can be accomplished by a covalent linkage between the support and the polynucleotide. The linkage unit, or linker, is designed to be stable and facilitate accessibility of the immobilized nucleic acid to a second genetic element to which it will be coupled. Alternatively, non-covalent linkages such as between biotin and avidin or stepavidin are useful. Such linkages have been used to create cDNA libraries on a solid support (Roeder (1998) Nucleic Acids Res. 26: 3451-3452). A typical method for attaching oligonucleotides is coupling a thiol functionalized polystyrene bead with a 3′ thiol-oligonucleotide under mild oxidizing conditions to form a disulfide linker. Examples of other functional group linkers include ester, amide, carbamate, urea, sulfonate, ether, and thioester. A primary amine group on a nucleic acid can be immobilized to carboxyl-derivatized beads using carbodiimide as the coupling reagent. A 5′ or 3′ biotinylated oligonucleotide can be immobilized on avidin or strepavidin bound to a support such as glass, sepharose (Pharmacia Biotech, Piscataway, N.J.), or microtiter plates (Pierce, Rockford, Ill.).

The directionality of the assembled polynucleotide and the component oligonucleotides coupled to the solid support may be in the 5′ or 3′ direction and both may be equally accommodated and efficient.

Typically, the immobilized polynucleotides are on a single solid support, e.g., immobilized to a single tube such that the immobilized polynucleotides are non-adressable. Thus, the polynucleotides are immobilized at random locations and each incoming nucleic acid molecule has an equal probability of contacting any of the immobilized polynucleotide molecules.

### Ligation

Sequential order of addition in the ligation reactions is a useful benefit of the present invention. In a ligation reaction, a ligation reagent affects ligation of DNA ends of the genetic elements to be coupled. DNA ligase conducts enzymatic ligation upon adjacent DNA ends to create an internucleotide phosphodiester bond and create a continuous strand in the immobilized ligation product. Ligation with DNA ligase is highly specific. With ATP or NAD⁺, DNA ligase catalyzes the formation of a phosphodiester bond between the 5′ phosphoryl terminus and the 3′-hydroxyl terminus of two, double-stranded oligonucleotides.

Polynucleotides can also be chemically ligated with reagents, such as cyanogen bromide and dicyclohexylcarbodiimide, to form an internucleotide phosphate linkage between two oligonucleotides, one of which bears a 5′ or 3′ phosphate group, annealed to a bridging oligonucleotide (Shabarova, et. al. Nucleic Acids Res. (1991) 19: 4247-51).

### Blocking and Deblocking

Often, a method is used to block and deblock the immobilized as well as incoming polynucleotides. This step prevents concatemer formation of incoming polynucleotides, as well as to allow only one coupling per immobilized polynucleotide during each round of ligation. The immobilized polynucleotide is deblocked such that it can be coupled to an incoming polynucleotide in a ligation reaction. Further, it is optimal that the incoming polynucleotide is blocked such that concatemers of the incoming polynucleotide do not form during the ligation reaction. One method to block and de-block is through the phosphorylation state of the immobilized and incoming genetic elements. An unphosphorylated 5′ end can be efficiently phosphorylated by a kinase, e.g., T4 polynucleotide kinase, in order to de-block an end. Similarly, a phosphatase can be used to remove a phosphate from an end in order to block the end from being ligated.

In a ligation reaction occuring at two double-stranded DNA ends, only one 5′ end needs to be phosphorylated for efficient ligation. In such a case, the ends are joined and the nick that is present in the opposite strand can later be sealed by propagation in a host cell like E. coli. Because only one phosphate group is necessary for ligation, changing the phosphorylation status of the immobilized polynucleotide provides a convenient means to block and debock.

The processes of ligation, de-blocking, and addition of incoming polynucleotide can be repeated any number of times to achieve construction of the desired genetic element.

### Amplification

Optionally, the library created on the solid support may be amplified directly from the solid support (i.e., using a method such as the polymerase chain reaction, with the immobilized nucleic acid as a template). In this case, the amplified DNA will not be coupled to the solid support, and can be removed from the solid support without enzymatic or chemical cleavage. Additionally, libraries of diverse molecules created on the solid support can be amplified using primers complementary to the first and last nucleic acids immobilized, and hance will amplify the entire library even if significant diversity was introduced in the coupling events.

### Cleavage

Optionally, the immobilized polynucleotides can be cleaved from the solid support. Cleavage can occur by enzymatic or chemical means. Often, cleavage is achieved using a restriction enzyme that recognizes a sequence at which to cleave in the immobilized polynucleotide. Cleavage can also occur by using an enzyme that recognizes a structure in a nucleic acid, like an apurinic site (i.e. by apurinic endonuclease), that is conveniently introduced at the desired site of cleavage.

### Religation

DNA ends may be rejoined covalently by incubating the DNA ends with an enzyme like a DNA ligase which will form phosphodiester bonds between nucleotides at the DNA end. Examples of ligases include E. coli DNA ligase, phage T4 DNA ligase, or human DNA ligases. These enzymes can be used under conditions well known to those skilled in the art to ligate DNA. Other enzymes are also capable of creating covalent linkages (like phosphodiester bonds) between nucleotides at DNA ends. Such enzymes include topoisomerases, transposons, integrases, and other recombination enzymes. Other mechanisms can be used to join DNA ends such as the utilization of an oligonucleotide whose sequence can hybridize to sequences on either end (i.e. both the 5′ and 3′ ends) to “bridge” the ends with hydrogen bonds (U.S. Pat. No. 5,942,609). The intervening sequence on the opposite strand may be filled in with a polymerase, such as E. coli polymerase, Klenow fragment, phage T4 polymerase, or Taq polymerase. Nicks may then be repaired by a DNA ligase as described above. Cellular extracts also contain ligase activities and cell or nuclear extracts could be used to rejoin DNA ends. Alternatively, DNA molecules could be introduced into intact cells and the cell's machinery could rejoin DNA ends by homologous or non-homologous means.

### Expression

In instance in which the genetic element immobilized to the solid support encodes a protein, expression of that protein may be accomplished by several different means. The immobilized genetic element, when it comprises a promotor, can be contacted with an RNA polymerase under appropriate conditions such that it transcribes the RNA encoded by the genetic element directly from the immobilized nucleic acid. Further, the RNA can be contacted with ribosomes and the relevant activated tRNAs, such that translation might occur. Indeed, in vitro transcription/translation kits are commercially available (Promega, Madison, Wis.) and could be applied directly to the immobilized nucleic acid.

Alternatively, the immobilized sequence may be cleaved from the solid support, and either expressed directly, e.g., in vitro, or introduced into a suitable host for expression; or cloned into a suitable vector for expression in the desired host. Further, it may be cleaved, recircularized, then exposed to in vitro transcription/translation reagents as described above.

### Uses of the Invention

It is contemplated that the present invention will have several uses as will be apparent to those of skill in the art. One use applies generally to the cloning of DNA. As it is difficult to couple several DNA fragments in solution to achieve efficient coupling of a relevant product, the present invention allows rapid, efficient, and specific order related coupling of several DNA fragments. These fragments can encode relevant features of expression vectors, various domains of proteins, or any other ordered arrangement of genetic elements desired by a researcher.

### Antibody Discovery

The invention is particularly useful for antibody discovery. This application involves the de novo synthesis of antibody genes from their component gene segments (i.e. V, D, and J segments) in vitro.

Antibody genes are comprised of gene segments. These segments are given the terms variable (abbreviated “V”), diversity (abbreviated “D”), junctional (abbreviated “J”), and constant (abbreviated “C”). There are two polypeptides of which an antibody is comprised: a light chain and a heavy chain. The polypeptide of a heavy chain is encoded by DNA comprised of V, D, J, and C genetic elements. A light chain polypeptide is encoded by DNA comprised of V, J, and C genetic elements. There are several V, D, and J segments comprising the antibody locus in the germline from which a rearranged functional gene may be derived. The combinatorial association of the various segments with one another in different B cells accounts for the enormous diversity of the immune system.

### Additional Applications

Another application is in the combinatorial construction of expression vectors in vitro. In this case, several different genetic elements such as promoters, enhancers, introns, may be coupled to genes of interest such that different promotors drive gene expression in the context of different enhancers, introns or other genetic elements.

Another application is in the field of molecular evolution. Current molecular evolution techniques require homology based PCR methods. The present invention would allow domain swapping, loop swapping, exon shuffling, or any other method of modular combinatorial gene construction. This method could be applied to create large libraries of combinatorially produced genes for screening to identify novel or improved genes or proteins.

Another example is the “humanization” of non-human proteins for therapeutic uses. It is well established that non-human proteins can stimulate immune responses when used as therapeutics. The current technology allows for the replacement of human domains in non-human proteins so that antigenicity may be minimized. This can be done by replacing “modules” or structural folds of non-human proteins with corresponding human counterparts. Of importance is the ability to make large libraries of combinatorally inserted human sequences into the non-human gene of interest.

The following examples are provided for illustration purposes and are not to be construed as a limitation on the invention.

### EXAMPLES

### Example 1

### Construction of a Double-stranded Nucleic Acid on a Solid Support

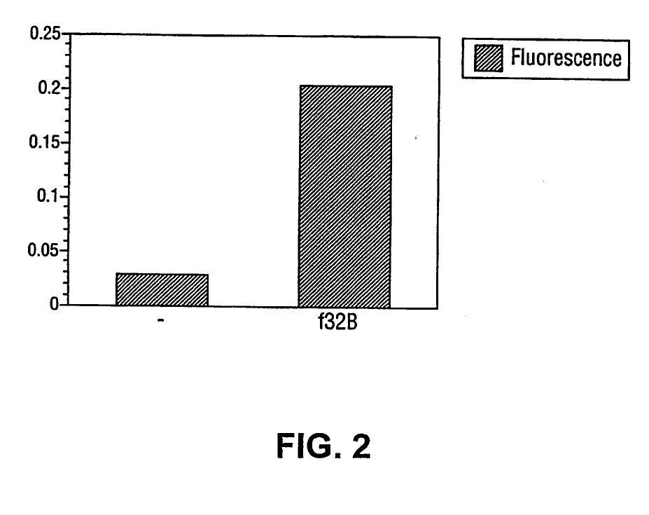

The present invention requires the attachment of a first genetic element to a solid support. As an example, FIG. 2 shows the adsorbance of a biotinylated 32 basepair double-stranded DNA fragment to a solid support comprised of a streptavidin coated microtiter plate well. In the first bar, no biotinylated DNA is added to the well, and in the second bar 200 ng of the 32-mer were added for 10 minutes, washed with TEN buffer (10 mM Tris-Cl pH 7.4, 1.0 mM EDTA, 100 mM NaCl) three times, and stained with picogreen dye (Molecular Probes, Eugene, Oreg.) prior to analysis on a spectrofluorometer. Other solid supports and DNA species can be used in the present invention, as described in the “detailed description” section.

### Ligation

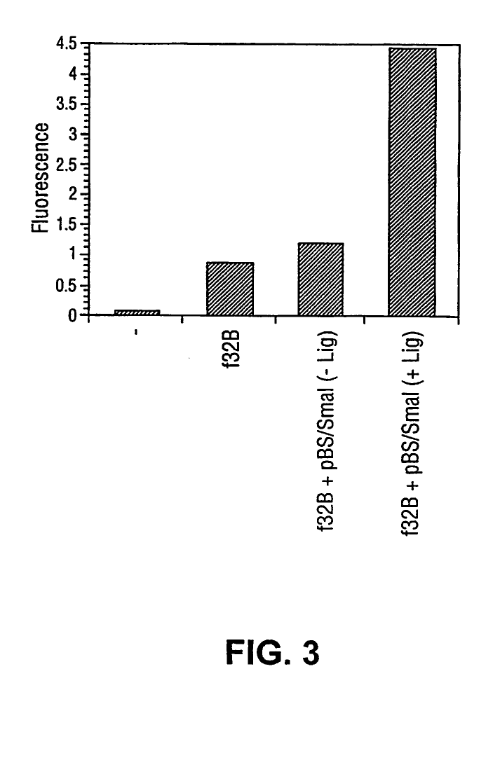

In order to determine whether a ligation reaction can be carried out while a DNA molecule is attached to a solid support, the 32-mer bound to streptavidin coated microtiter wells was exposed to the plasmid pBluescript SK II—linearized with Sma I in the presence (FIG. 3, bar 4) or absence (FIG. 3, bar 3) of T4 DNA ligase. Following the ligation reaction, the solid support was washed extensively with TEN buffer to remove non-specifically bound DNA. As can be seen, the presence of pBluescript and DNA ligase produces an increase in fluorescent signal produced by the picogreen dye.

### Cleavage

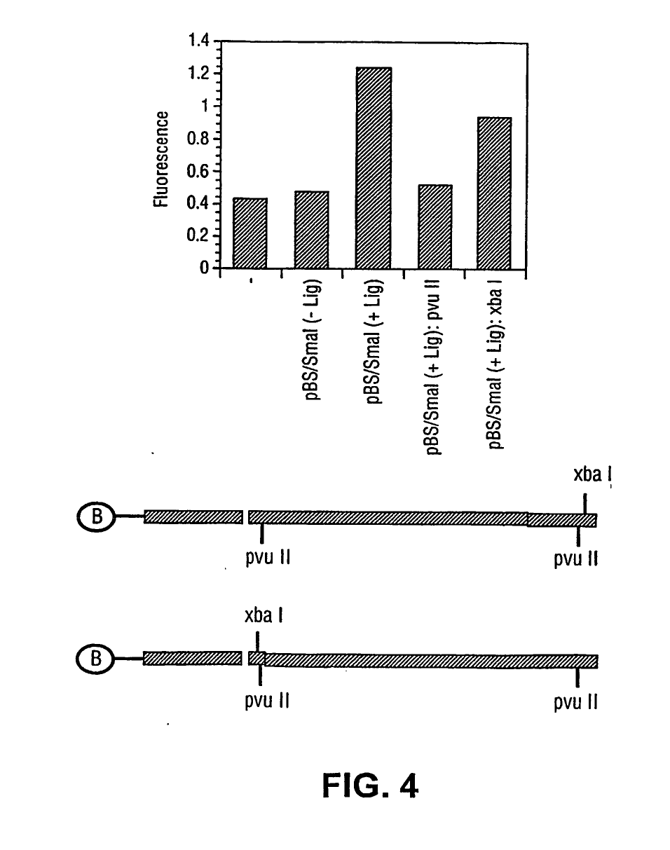

Cleavage from the solid support can be accomplished through the use of a restriction enzyme as shown in FIG. 4. In this example, two restriction sites for the enzyme pvu II exist in the pBluescript vector, such that the entire vector will be cleaved (FIG. 4, bar 4). The enzyme xba I, however, is only located in one location, such that cleavage is dependent on the orientation of the initial ligation. Thus, in FIG. 4 bar 5, only 50% of the plasmid is cleaved from the solid support, since the original ligation could have occurred in either of two orientations.

### Phosphate Requirement

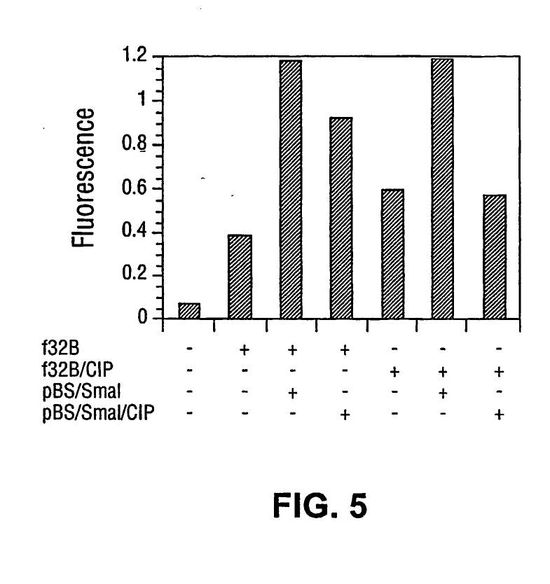

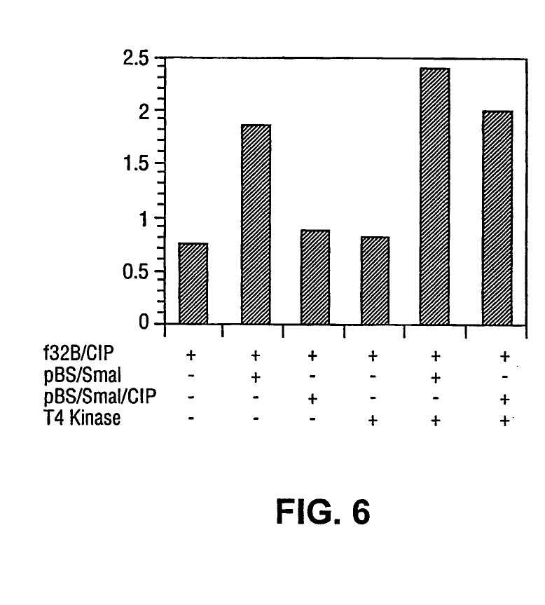

The success of solid support mediated cloning requires some process to block and de-block. This requirement ensures that genetic elements added to the solid support do not become ligated to one another, forming concatemers in solution. Additionally it ensures that only one genetic element is coupled to the solid support per round of ligation. One mechanism to block ligation in solution is to remove the phosphates from the 5′ end of the incoming genetic elements. Thus, FIG. 5 illustrates that at least one phosphate is required on the immobilized DNA for ligation to occur. Bar 4 shows that dephosphorylated incoming DNA can be ligated to immobilized DNA. However, bar 7 shows that dephosphorylated immobilized DNA does not ligate to dephosphorylated incoming DNA. Thus, when an incoming DNA molecule is ligated to an immobilized DNA molecule, it must then be kinased in order to phosphorylate the end so that it will be available to ligate to an incoming DNA fragment. FIG. 6 shows that T4 kinase can serve to phosphorylate the immobilized DNA such that it is available to ligate to incoming DNA (compare bar 3 and bar 6 in FIG. 6). Therefore, rounds of ligation and phosphorylation can serve to block and de-block the growing DNA molecule immobilized on the solid-support.

### Libraries

The double-stranded blunt ended oligonucleotide f50-amino (SEQ) is covalently coupled to polystyrene encapsulated paramagnetic microspheres according to the manufacturer's instructions (Bangs Laboratories, Fisher, Ind.). The f50-conjugated microspheres are incubated with a solution containing 1% bovine serum albumin, 0.025% tween-20, 10 mM Tris-Cl, and 1 mM EDTA (BTTE) for 1 hour at 25° C. Solution is removed by applying a strong magnet to the microfuge tube and aspirating the solution with a pipette tip. The f50-conjugated microspheres are then washed twice in 1× ligase buffer (Invitrogen, Carlsbad, Calif.). The plasmid pBluscript II SK—was linearized by S1 nuclease, which cleaves supercoiled plasmids at a single random position (WO02/16642) and produces a population of linear DNA molecules containing molecules of approximately equal length but having different DNA sequences at the ends. This population of randomly cleaved plasmid is gel purified on a 1.5% agarose gel by using Qiex beads according to the manufacturers instructions (Qiagen, Chatworth, Calif.). This population of DNA is then dephosphorylated using calf intestinal phosphatase (1 unit/μg DNA in a 20 μl reaction) for 5 minutes. The phosphatase is inactivated by heating the sample to 70° C. for 5 minutes. The 1 μg of dephosphorylated plasmid is added to 1 mg of f50 conjugated encapsulated paramagnetic microspheres in 1× ligase buffer (50 μl), followed by addition of 400 units of T4 DNA ligase (New England BioLabs, Beverly, Mass.). This reaction is incubated for 10 minutes at 37° C.

### Example 2

### De Novo Prepration of an Antibody Gene

Antibody genes are comprised of gene segments. These segments are given the terms variable (abbreviated “V”), diversity (abbreviated “D”), junctional (abbreviated “J”), and constant (abbreviated “C”). There are two polypeptides of which an antibody is comprised: a light chain and a heavy chain. The polypeptide of a heavy chain is encoded by DNA comprised of V, D, J, and C genetic elements. A light chain polypeptide is encoded by DNA comprised of V, J, and C genetic elements. There are several V, D, and J segments comprising the antibody locus in the germline from which a rearranged functional gene may be derived. The combinatorial association of the various segments with one another in different B cells accounts for the enormous diversity of the immune system.

To create a library of human antibody light chains, a double-stranded 50 basepair oligonucleotide containing a primary amine group attached to its 5′ end and a 3′ overhang of CAGC (This overhang can be used as on half of a SfiI restriction enzyme site) is covalently coupled to polystyrene encapsulated paramagnetic particles derivitized by a carboxyl group according to the manufacturers instructions (Bangs Laboratories, Fisher, Ind.). DNA is amplified from the Kappa II group of human antibody light chain V regions by the polymerase chain reaction using the forward primer mix (Sfi I site is in italics, degenerate bases at a position are in parentheses) AAGTCTGTGCCCCTAA GGCCCAGCCGGCC GAT (A/G)TT GTG ATG AC(C/T) CAG (A/T)CT CCA, and the reverse primer mix GG AGG (A/C)(A/C)(G/A) GTG T(G/A)T ACC TTG CAT, which will amplify at least four of the Kappa II group of V regions. The amplification reaction consists of 100 ng human genomic DNA, 1 μM of each primer, 1 μl of pfu polymerase in the reaction buffer provided by the supplier (Stratagene, LaJolla, Calif.), and 100 μM dNTPs. Following 30 cycles of hot-started amplification with denaturation at 94° C. for 30 seconds, annealing at 56° C. for 30 seconds, and extension at 72° C. for 30 seconds, the 300 basepair product is desalted, digested with Sfi I, dephosphorylated with calf intestinal phosphatase, then gel purified on a 1% agarose gel. The digested DNA is added to the DNA-paramagnetic particles and incubated in the presence of 400 units of T4 DNA ligase in DNA ligase buffer (New England Biolabs, Beverly, Mass.) for 10 minutes at room temperature. A magnet is applied to the side of the tube, and the supernatant removed. The particles are washed twice with BTTE, and then a mixture of BsiWI digested double-stranded unphosphorylated oligonucleotides encoding human the human J region (all are around 50 nucleotides in length; the sequences can be found at http://www.mrc-cpe.cam.ac.uk/) with a BsiWI site in frame at the 3′ end are added to the particles in 50 μl of ligase buffer and 1 μl of T4 DNA ligase. Following this round of ligation, a magnet is applied and the supernatant removed is removed followed by washing of the beads twice with BTTE. A subsquent round of ligation is then carried out at the above conditions with plasmid pDcK linearized with BsiWI and SfiI, and dephosphorylated with calf intestinal phosphatase. Following ligation, the beads are again washed with BTTE, and treated with SfiI to release the plasmid. The linear plasmid is then subjected to circularization with T4 DNA ligase to form the antibody light chain library in solution.

### Example 3

### Molecular Evolution—Kringle Domain Containing Proteins

The field of molecular evolution is concerned with the optimization or alteration of genes and proteins. Generally molecular evolution strategies involve the mutagenesis of a gene, or family of genes of interest. In the currently most robust technique, DNA shuffling, a family of related genes are fragmented, denatured, annealed, and extended with polymerase to produce a library of hybrid genes. This library is then screened for a function of interest to identify more optimal sequences. The process can then be repeated recursively. DNA shuffling has proven effective in molecular evolution, but suffers from its requirement for significant homology at the step of annealing. The ability to eliminate homology requirements would allow vastly more sequence space to be explored in the construction of genetic evolution libraries. However, the ability to eliminate homology requirements coupled with the ability to utilize fragments which encode structurally similar protein domains, could lead to significant strides in molecular evolution technology.

Several protein classes contain ordered domain structures with low homology. One such class of proteins contains multiple “Kringle” domains. Kringle domain containing proteins include tissue plasminogen activator (tPA), urokinase, plasminogen, hepatocyte growth factor, prothrombin and Apo(a) (Wu, et. al. Proc. Natl. Acad. Sci (1997) 94: 13654-13660; Wisdedt, et. al. J. Biol. Chem. (1998) 273: 24420-24424; Kuba, et. al. Cancer Res. (2000) 60: 6737-6743). These proteins are medically important as drugs or drug targets. Fragmants of some of these proteins containing certain kringle domains contain potent and unique properties compared to the parent molecule. For example, the antiangiogenic protein angiostatin, is a derivative of plasminogen and has potent antitumor effects (Cao, et. al. J. Biol. Chem. (1996) 271: 29461-29467; Ji, et. al. FASEB J (1998) 12: 1731-1738; Cao, et. al. Proc. Natl. Acad. Sci (1999) 96: 5728-5733). Additionally, fragments of hepatocyte growth factor (HGF) can inhibit HGF itself, but also has an additional antiangiogenic property (Kuba, et. al. Cancer Res. (2000) 60: 6737-6743). Thus, combinatorally rearranging and combining kringle domains is a strategy for identifying new protein drugs.

A combinatorial library of kringle domain containing proteins can be made by combining the nucleic acids encoding kringle domains of the above proteins and recursively coupling such mixtures in a stepwise fashion on a solid support, as described above in earlier examples. The nucleic acids for the kringle domains are amplified as described above for antibody V regions, but with primers designed to hybridize to each individual kringle domain in separate PCR reactions. The primers are designed to contain unique restriction sites at each end to facilitate directional coupling. The individual PCR reactions are combined into a single mixture. This mixture is used to recursively couple to an immobilized DNA fragment to produce a library containing 3, 4, 5, or 6 kringle domains. The library is then ligated into an expression vector and expressed to identify novel kringle domain containing proteins using one of several assays known in the art, for example to investigate cell proliferation, migration, angiogenesis, or protease cleavage enhancement or inhibition.

All publications, patents, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of preparing a library of double-stranded polynucleotides, each of which comprises a multiplicity of genetics elements, the method comprising:
providing a first population of double-stranded polynucleotides, wherein the first population is immobilized to a solid support in a non-addressable configuration;
providing a second population of double-stranded polynucleotides, wherein the second population comprises a multiplicity of different polynucleotides encoding a genetic element;
and covalently coupling the second population to the first population to create a library of double-stranded polynucleotides in a non-addressable configuration.

2. The method of claim 1, wherein the first population of polynucleotides comprises a multiplicity of different sequences encoding a genetic element.

3. The method of claim 1, further comprising a step of covalently coupling a third population of double-stranded polynucleotides to the immobilized sequences comprising the first and second populations.

4. The method of claim 3, wherein the third population comprises a multiplicity of different double-stranded polynucleotides encoding a genetic element.

5. The method of claim 2, wherein the genetic element encoded by the first population comprises a promoter or enhancer sequence.

6. The method of claim 2, wherein the genetic element encoded by the first population comprises a polypeptide domain.

7. The method of claim 6, wherein the polypeptide domain is selected from the group consisting of an antibody V, D, or J segment.

8. The method of claim 7, wherein the antibody segment is a V segment.

9. The method of claim 8, wherein the genetic element encoded by the second population encodes a J segment of an antibody gene.

10. The method of claim 6, wherein the polypeptide domain is a kringle domain.

11. The method of claim 6, wherein the polypeptide domain is encoded by a fragment of a carbon-carbon lyase gene.

12. The method of claim 6, wherein the polypeptide domain is selected from the group consisting of α-helices, β-strands, β-sheets, β-turns, and loops.

13. The method of claim 12, wherein the second population encodes a polypeptide domain that is selected from the group consisting of α-helices, β-strands, β-sheets, β-turns, and loops.

14. The method of claim 1, wherein the second polynucleotide population is blocked.

15. The method of claim 14, wherein the second polynucleotide population is blocked by dephosphorylating the ends of the double-stranded oligonucleotide.

16. The method of claim 1, further comprising a step of deblocking the immobilized polynucleotides.

17. The method of claim 16, wherein the step of deblocking the immobilized polynucleotides comprises phosphorylating the 5′ end.

18. The method of claim 1, further comprising a step of transcribing and translating the immobilized polynucleotide.

19. The method of claim 1, further comprising a step of cleaving the immobilized polynucleotides from the solid support.

20. The method of claim 19, further comprising a step of ligating the cleaved population of polynucleotides to produce a population of circular polynucleotides.

21. The method of claim 1, further comprising a step of selecting a member of the double-stranded polynucleotide library that has a desired function.

22. A library of double-stranded nucleic acids prepared using the method of claim 1.

23. A library of immobilized double-stranded nucleic acids, each of which encodes a multiplicity of genetic elements, wherein the immobilized nucleic acids are in a non-addressable configuration.
